(19) United States
(12) Patent Application Publication
(10) Pub. No.: US 2026/0083328 A1
(43) Pub. Date: Mar. 26, 2026

(54) IMAGE ALIGNMENT DEVICES AND ASSOCIATED METHODS

(71) Applicant: ARTHREX, INC., Naples, FL (US)

(72) Inventors: Nick Metcalfe, Bonita Springs, FL (US); Scott William Doody, Bonita Springs, FL (US); Shervin Kazemi, Naples, FL (US)

(21) Appl. No.: 19/324,837

(22) Filed: Sep. 10, 2025

Related U.S. Application Data
(60) Provisional application No. 63/698,612, filed on Sep. 25, 2024.

(51) Int. Cl. A61B 5/00; A61B 17/15; A61B 17/17

(52) U.S. Cl. CPC A61B 5/0033; A61B 5/0071; A61B 17/15; A61B 17/1703; A61B 2560/0233

(57) ABSTRACT

This disclosure relates to surgical systems, devices and methods for planning and implementing surgical procedures. The systems and methods disclosed herein may be utilized for surgical imaging, including alignment of an imaging device relative to the anatomy of a patient.

### CROSS-REFERENCE TO RELATED APPLICATIONS

This application claims the benefit of U.S. Provisional Application No. 63/698,612, filed Sep. 25, 2024, which is incorporated herein by reference in its entirety.

### BACKGROUND

This disclosure relates to surgical systems and methods for planning and implementing surgical procedures, including devices useful for aligning imaging devices relative to patient anatomy.

Many bones of the human musculoskeletal system include articular surfaces. The articular surfaces articulate relative to other bones to facilitate different types and degrees of joint movement. The articular surfaces can erode or experience bone loss over time due to repeated use or wear or can fracture due to a traumatic impact. These types of bone defects can cause joint instability and pain. A prosthesis may be implanted in the patient to restore functionality to the joint. One or more guides may be used to remove bone prior to implanting the prosthesis. A surgeon or clinical user may use a camera to capture one or more images of the anatomy for confirming alignment of the guide relative to the anatomy prior to bone removal.

### SUMMARY

This disclosure relates to systems, devices and methods of performing a surgical procedure. The systems may be utilized for positioning an imaging device relative to the anatomy of a patient.

An alignment guide for surgical imaging may include a guide body including first and second sidewalls interconnecting proximal and distal portions to bound a cavity. The proximal portion may include a foreground sight. The distal portion may include a background sight. The foreground sight may be visually alignable with the background sight to indicate an orientation of the alignment guide relative to a field of view of an imaging device. A first alignment opening may extend along a first reference plane between the first sidewall and the cavity. A second alignment opening may extend along a second reference plane between the second sidewall and the cavity. The first and second reference planes may be transverse to each other and may intersect along the background sight.

A surgical kit may include an instrument guide including one or more apertures dimensioned to guide a surgical device. The instrument guide may be adapted to contact an anatomical surface of a patient based on a surgical plan. An alignment guide may be securable to the instrument guide at one or more interfaces. The alignment guide may include a guide body including a cavity. A foreground sight and a background sight may be on opposite sides of the cavity. First and second alignment openings may be interconnected by the cavity. The first and second alignment openings may extend along respective reference planes that may intersect at the foreground and background sights to establish respective pathways for a set of laser beams projectable onto the background sight. The foreground and background sights may be visually alignable with each other to indicate an orientation of the instrument guide relative to a field of view of an imaging device.

A system for surgical imaging may include an imaging device including a set of lasers. The lasers may be operable to emit laser beams along respective paths oblique to a field of view of the imaging device such that the laser beams may intersect each other adjacent to an optical center of the field of view. An alignment guide may include a radiopaque guide body. A foreground sight and a background sight may be visually alignable with each other to indicate an orientation of the guide body relative to the field of view of the imaging device. A set of alignment openings may be along an exterior of the guide body. The alignment openings may extend along respective reference planes that may intersect the foreground sight and the background sight. The alignment openings may be dimensioned to permit a projection of the respective laser beams onto the background sight.

A method for a surgical imaging procedure may include positioning an anatomy of a patient relative to an imaging device. The imaging device may include a set of lasers. The set of lasers may be operable to emit laser beams along respective paths oblique to a field of view of the imaging device. The method may include setting a position of an alignment guide relative to the anatomy. The alignment guide may include a foreground sight and a background sight opposed to each other. The method may include projecting, from the set of lasers, laser beams along the respective paths such that the laser beams may intersect each other at an optical center of the field of view. The method may include setting a capture position of the imaging device relative to the alignment guide such that the foreground sight and the background sight may be substantially aligned with each other relative to the field of view of the imaging device and such that the laser beams may intersect each other along the background sight. The method may include capturing, using the imaging device, an image at the capture position.

The present disclosure may include any one or more of the individual features disclosed above and/or below alone or in any combination thereof.

The various features and advantages of this disclosure will become apparent to those skilled in the art from the following detailed description. The drawings that accompany the detailed description can be briefly described as follows.

### BRIEF DESCRIPTION OF THE DRAWINGS

FIG. 1 discloses a perspective view of an alignment device.

FIG. 2 discloses a proximal view of the alignment device of FIG. 1.

FIG. 3 discloses a side view of the alignment device of FIG. 1.

FIG. 4 discloses a section view of the alignment device taken along line 4-4 of FIG. 2.

FIG. 5 discloses a section view of the alignment device taken along line 5-5 of FIG. 3.

FIGS. 6-8 discloses the alignment device at a first orientation relative to a surgical instrument.

FIGS. 9-11 discloses the alignment device at a second orientation relative to the surgical instrument of FIGS. 6-8.

FIG. 12 disclose orientations of an axis of the alignment device.

FIG. 13 discloses a perspective view of an alignment device according to another implementation.

FIGS. 14-16 disclose the alignment device of FIG. 13 at a first orientation relative to an anatomy.

FIGS. 17-18 disclose the alignment device of FIG. 13 at a second orientation relative to an anatomy.

FIG. 19 discloses an alignment device according to another implementation.

FIGS. 20-21 disclose an alignment device and anatomy relative an imaging system.

FIG. 22 discloses a proximal view of the alignment device of FIGS. 20-21.

FIGS. 23-24 disclose images of an implementation of the imaging system of FIGS. 20-21.

FIG. 25 discloses a method of surgical imaging in a flowchart according to an implementation.

FIG. 26 discloses an image of an alignment device relative to a first anatomical plane of the anatomy.

FIG. 27 discloses virtual depiction of the alignment device relative to the first anatomical plane of the anatomy.

FIG. 28 discloses an image of the alignment device relative to a second anatomical plane of the anatomy.

FIG. 29 discloses virtual depiction of the alignment device relative to the second anatomical plane of the anatomy.

FIG. 30 discloses an image of an alignment device with respect to one or more dimensions.

FIG. 31 discloses a virtual depiction of the alignment device with respect to the one or more dimensions of FIG. 30.

FIG. 32 discloses an alignment device according to another implementation.

Like reference numbers and designations in the various drawings indicate like elements.

### DETAILED DESCRIPTION

This disclosure relates to surgical systems, devices and methods for aligning imaging devices relative to patient anatomy. Image alignment devices and associated methods of capturing images of anatomy are disclosed.

Orthopaedic and other surgical procedures may utilize one or more transfer devices to position various surgical devices relative to the patient anatomy. The transfer guides may be patient-specific, configurable (e.g., calibrated) and/or standard (e.g., fixed and non-patient specific). The transfer devices may include cut (e.g., drill or resection) guides and other surgical instruments. During the process of performing surgery involving any sort of patient matched or geometrically calibrated transfer device, significant amounts of (e.g., fluoroscopic) images may be required for device placement verification. The transfer device may be utilized to confirm that the anatomy and/or surgical device(s) are substantially perpendicular to a field of view of the imaging device. The transfer device may be adapted to align the patient anatomy in a planar view created by the fluoroscopic image. The disclosed techniques may be utilized to reduce the amount of imaging required and/or reduce the time of the operation. The disclosed techniques may be utilized to improve the accuracy and validation of placement of transfer devices according to surgical plans established for patients. The disclosed techniques may be utilized to verify that a patient-specific surgical instrument may be correctly positioned on the anatomy.

The (e.g., image) alignment devices (e.g., guides) and associated techniques disclosed herein may be utilized to (e.g., visually) align the anatomy. The anatomy may be aligned relative to an imaging device based on a depth of the view (e.g., foreground and background) associated with the alignment device. The anatomy may be aligned relative to the imaging device based on one or more (e.g., laser) beams, which may be emitted adjacent to a camera of the imaging device. The alignment devices may be useful for various procedures that use fluoroscopic alignment. The alignment device may be a separate component or may be incorporated into another surgical device.

Methods of surgical imaging may include aligning an imaging device relative to the patient anatomy. Various imaging devices may be utilized to capture imagery of the anatomy, including fluoroscopic, computed tomography (CT), magnetic resonance imaging (MRI) and/or ultrasound devices. The imaging device may include a (e.g., fluoroscopic) camera. The imaging device may include light emitting device(s) such as one or more lasers. The lasers may be arranged relative to the camera. Laser beams emitted by the lasers may be oblique to a field of view of the camera. The lasers may be arranged such that the laser beams may intersect at an optical center of the camera. The laser beams may intersect to a establish a crosshair. The surgeon or clinical user may position an alignment device (e.g., guide) relative to the patient anatomy. The alignment device may be secured to a surgical instrument or other device, including a cut guide and/or one or more guide elements. The alignment device may include a foreground sight and/or a background sight. A capture position of the of the camera may be adjusted or otherwise set, which may include moving the alignment device and anatomy relative to each other. The foreground and background sights may be arranged relative to each other within the camera field of view to align the anatomy based on a depth of the view. The foreground and background sights may be aligned along an axis, which may be substantially perpendicular to the field of view. The lasers may intersect along a surface of the background sight and/or other portions of the alignment device for alignment relative to the optical center of the camera. The disclosed techniques may be utilized to confirm foreground and background alignment and may be used to (e.g., simultaneously) align the lasers to the alignment guide to obtain confirmation that the anatomy is substantially normal to the camera field of view.

Images may be captured relative to the anatomy, including relative to one or more anatomical planes of the patient. The images may be captured to verify a position and orientation of the surgical instrument prior to removal of bone and other tissue for subsequent placement of one or more implant components. The removal may be performed using one or more drills, saw blades and/or resection tools. The alignment device may be utilized to set a position of the imaging device relative to the anatomical plane(s). The captured image(s) may be compared to virtual depiction(s) of the anatomy and alignment device to verify alignment. The virtual depiction(s) may be included or otherwise associated with the surgical plan. The disclosed techniques may be utilized to confirm that the image (e.g., coronal, sagittal or transverse) view to be captured may be representative of the virtual depiction. The imaging device and alignment device may be repositioned relative to the anatomy (e.g., moved approximately 90 degrees). One or more additional images of the anatomy may be captured utilizing any of the techniques disclosed herein.

An alignment guide for surgical imaging may include a guide body including first and second sidewalls interconnecting proximal and distal portions to bound a cavity. The proximal portion may include a foreground sight. The distal portion may include a background sight. The foreground sight may be visually alignable with the background sight to indicate an orientation of the alignment guide relative to a field of view of an imaging device. A first alignment opening may extend along a first reference plane between the first sidewall and the cavity. A second alignment opening may extend along a second reference plane between the second sidewall and the cavity. The first and second reference planes may be transverse to each other and may intersect along the background sight.

In any implementations, the guide body may be securable to an instrument guide.

In any implementations, the first and second reference planes may be substantially perpendicular to each other.

In any implementations, the first and second alignment openings may extend from a proximal face of the proximal portion. An arc shaped channel may extend about a periphery of the foreground sight to interconnect the first and second alignment openings along the proximal face.

In any implementations, the background sight may include first and second alignment members that may intersect at a junction to establish a cross-shaped geometry.

In any implementations, the first and second reference planes may follow the respective first and second alignment members and may intersect the junction.

In any implementations, the foreground sight may include an elliptical body that may be situated in an elliptical opening along a proximal face of the proximal portion. The elliptical body may include a cross-shaped opening that may be alignable with a central portion of the background sight.

In any implementations, the first and second reference planes may intersect along the cross-shaped opening and the central portion.

In any implementations, one or more guide passages may be dimensioned to receive a respective guide element.

In any implementations, the one or more guide passages may include first and second guide passages that may extend along respective passage axes. The passage axes may be substantially parallel to each other.

In any implementations, the first and second guide passages may be defined in respective flanges that may extend from the guide body.

In any implementations, the one or more guide passages may include first and second guide passages. A bridge member may interconnect the guide body and a locating member. The bridge member may be dimensioned to span across a joint of an anatomy. The first guide passage may be established in the guide body. The second guide passage may be established in the locating member.

In any implementations, the guide body may be monolithic.

In any implementations, the guide body may include a metallic material.

A surgical kit may include an instrument guide including one or more apertures dimensioned to guide a surgical device. The instrument guide may be adapted to contact an anatomical surface of a patient based on a surgical plan. An alignment guide may be securable to the instrument guide at one or more interfaces. The alignment guide may include a guide body including a cavity. A foreground sight and a background sight may be on opposite sides of the cavity. First and second alignment openings may be interconnected by the cavity. The first and second alignment openings may extend along respective reference planes that may intersect at the foreground and background sights to establish respective pathways for a set of laser beams projectable onto the background sight. The foreground and background sights may be visually alignable with each other to indicate an orientation of the instrument guide relative to a field of view of an imaging device.

In any implementations, the one or more interfaces may include a first interface and a second interface. The guide body may be situated at a first orientation relative to the instrument guide at the first interface. The guide body may be situated at a second orientation relative to the instrument guide at the second interface.

In any implementations, the foreground and background sights may be established along an axis of the guide body. A position of the axis associated with the first orientation may be substantially perpendicular to a position of the axis associated with the second orientation when projected onto a common reference plane.

In any implementations, the one or more interfaces may include a receptacle dimensioned to mate with a protrusion to orient the alignment guide at a preselected orientation relative to the instrument guide.

A system for surgical imaging may include an imaging device including a set of lasers. The lasers may be operable to emit laser beams along respective paths oblique to a field of view of the imaging device such that the laser beams may intersect each other adjacent to an optical center of the field of view. An alignment guide may include a radiopaque guide body. A foreground sight and a background sight may be visually alignable with each other to indicate an orientation of the guide body relative to the field of view of the imaging device. A set of alignment openings may be along an exterior of the guide body. The alignment openings may extend along respective reference planes that may intersect the foreground sight and the background sight. The alignment openings may be dimensioned to permit a projection of the respective laser beams onto the background sight.

In any implementations, the reference planes may be substantially perpendicular to each other. The reference planes may intersect adjacent to a center of the foreground sight and may intersect adjacent to a center of the background sight.

In any implementations, an instrument guide may be adapted to contact an anatomical surface of a patient based on a surgical plan. The alignment guide may be securable to the instrument guide to indicate an orientation of the instrument guide relative to the field of view of the imaging device.

In any implementations, the guide body may be radiopaque such that visibility of the background sight may be partially obstructed adjacent to the alignment openings.

In any implementations, the imaging device may include a fluoroscopic camera.

A method for a surgical imaging procedure may include positioning an anatomy of a patient relative to an imaging device. The imaging device may include a set of lasers. The set of lasers may be operable to emit laser beams along respective paths oblique to a field of view of the imaging device. The method may include setting a position of an alignment guide relative to the anatomy. The alignment guide may include a foreground sight and a background sight opposed to each other. The method may include projecting, from the set of lasers, laser beams along the respective paths such that the laser beams may intersect each other at an optical center of the field of view. The method may include setting a capture position of the imaging device relative to the alignment guide such that the foreground sight and the background sight may be substantially aligned with each other relative to the field of view of the imaging device and such that the laser beams may intersect each other along the background sight. The method may include capturing, using the imaging device, an image at the capture position.

In any implementations, the alignment guide may include a cavity bounded by the foreground sight and the background sight. The alignment guide may include a set of alignment openings distributed along an exterior of the alignment guide. The step of setting the capture position may include projecting the laser beams through the respective alignment openings onto the background sight.

In any implementations, the step of setting the capture position may include projecting the laser beams along respective alignment members of the background sight such that the laser beams may intersect each other at a junction between the alignment members.

In any implementations, the alignment openings may extend along respective reference planes that may be substantially perpendicular to each other. The reference planes may intersect the foreground sight and the background sight.

In any implementations, the method may include setting a positioning of an instrument guide along the anatomy. The step of setting the position of the alignment guide may include securing the alignment guide at a first interface of the instrument guide.

In any implementations, the instrument guide may be adapted to contact the anatomy at a patient-specific position based on a surgical plan associated with the patient.

In any implementations, the method may include securing the alignment guide at a second interface of the instrument guide, and then may include repeating the step of setting the capture position and the capturing step.

In any implementations, the captured images associated with the alignment guide at the first and second interfaces may correspond to different anatomical planes of the anatomy.

In any implementations, the anatomical planes may include a coronal plane and/or a sagittal plane of the anatomy.

In any implementations, the method may include guiding, using the instrument guide, at least one surgical device relative to the anatomy subsequent to the capturing step.

In any implementations, the step of setting the position of the alignment guide may include positioning the alignment guide on one or more guide elements positioned in the anatomy.

In any implementations, the method may include positioning, using the one or more guide elements, an implant component relative to the anatomy subsequent to the capturing step.

In any implementations, the step of setting the capture position may include comparing the captured image to a virtual depiction of the alignment guide relative to the anatomy associated with a surgical plan for the patient.

In any implementations, the step of comparing the captured image may include comparing a value of a dimension associated with the alignment guide and the anatomy in the captured image to a value of the dimension in the virtual depiction.

In any implementations, the alignment guide may be radiopaque.

In any implementations, the imaging device may include a fluoroscopic camera.

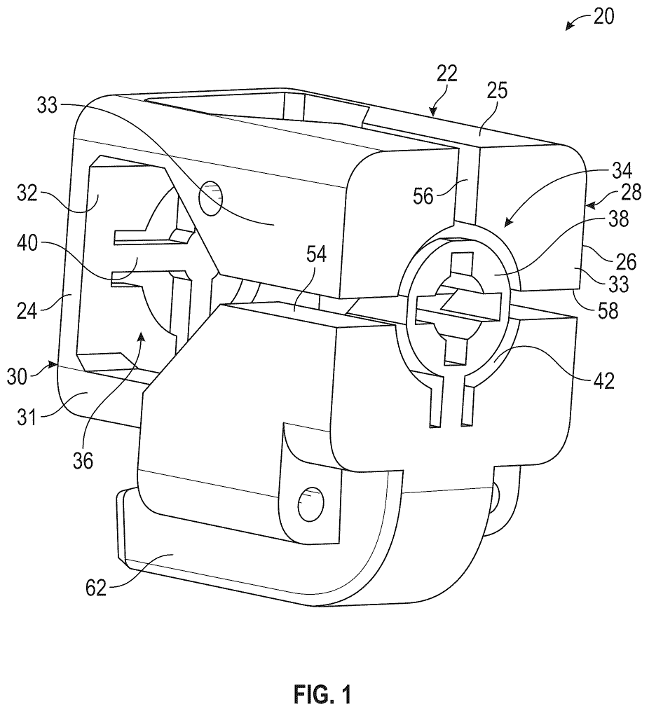

FIGS. 1-5 disclose an alignment device (e.g., guide) 20 according to an implementation. The alignment guide 20 may be utilized for surgical imaging, including alignment of an imaging device relative to an anatomy of a patient and/or one or more surgical devices. The anatomy may include one or more bones and/or joints of the patient. The bones may include one or more bones associated with a joint, including various long bones such as a humerus, femur, or tibia. The joints may include a shoulder, ankle, knee, hip or elbow joint. The surgical devices may include many include instrumentation and/or one or more implants positioned along the anatomy.

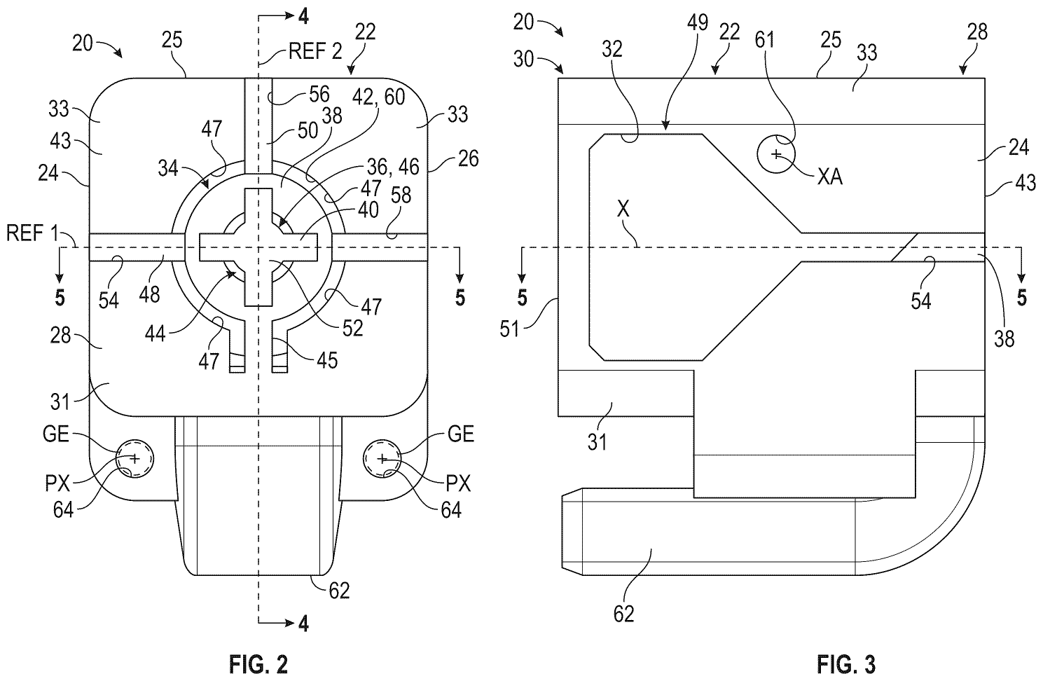

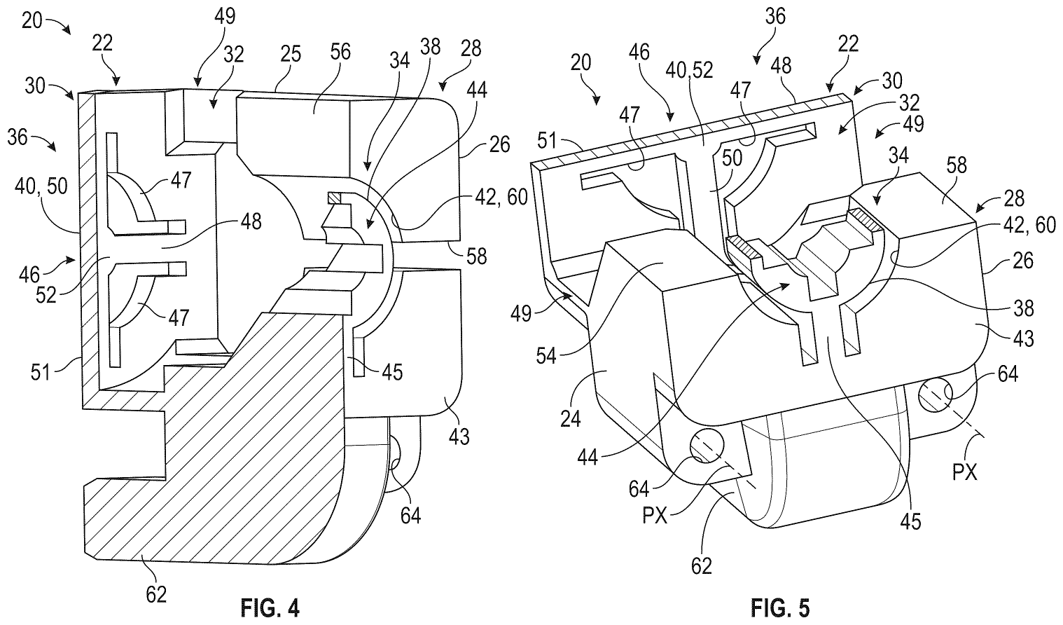

Referring to FIGS. 1-3, the alignment guide 20 may include a guide body 22. The guide body 22 may have a generally block-shaped geometry. The guide body 22 may be a monolithic structure. In other implementations, the guide body 22 may include one or more components fixedly attached or otherwise secured to each other. The guide body 22 may include one or more sidewalls. In the implementation of FIGS. 1-2, the guide body 22 may include first, second and/or sidewalls 24, 25, 26. The guide body 22 may be dimensioned to extend between a first (e.g., proximal) portion 28 and a second (e.g., distal) portion 30. The sidewalls 24, 25, 26 may be dimensioned to interconnect the proximal and distal portions 28, 30. The sidewalls 24, 25, 26 and/or proximal and distal portions 28, 30 may be dimensioned to bound a cavity 32. Aspects of the cavity 32 are disclosed in the sectional views of FIGS. 4-5. In implementations, the guide body 22 may include a base 31 and one or more (e.g., boundary) sections 33. The sections 33 may be dimensioned to extend from the distal portion 30 to a proximal face 43 of the proximal portion 28. In implementations, the sections 33 may be cantilevered from the distal portion 30 (e.g., FIGS. 1 and 3). Each section 33 may extend along the sidewall 25 and may extend along a respective one of the sidewalls 24, 26. The distal portion 30 may be dimensioned to interconnect the base 31 with the sections 33.

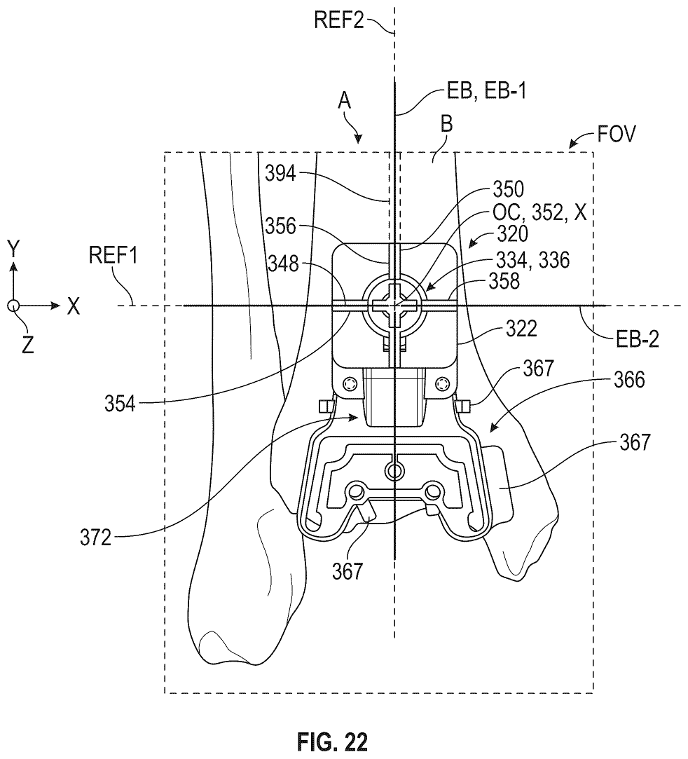

The alignment guide 20 may include one or more alignment features for aligning the anatomy and/or one or more surgical devices relative to an imaging device. Aligning the alignment features relative to each other and/or a field of view of the image device may be useful for capturing one or more images in which the alignment guide 20, surgical device(s) and/or anatomy may be substantially perpendicular to the field of view of the imaging device. In implementation of FIGS. 1-2, the alignment guide 20 may include a foreground (e.g., proximal or front) sight 34 and/or a background (e.g., distal or rear) sight 36. In implementations, the foreground sight 34 may be established along the proximal portion 28. The background sight 36 may be established along the distal portion 30. The foreground sight 34 and background sight 36 may be established along a (e.g., longitudinal) axis X of the guide body 22 (e.g., FIGS. 2-3). The foreground sight 34 and background sight 36 may be established on opposite sides of the cavity 32. The foreground sight 34 may be visually alignable with the background sight 36 to indicate an orientation of the alignment guide 20 (e.g., FIG. 2), including relative to a field of view of an imaging device (e.g., FIG. 22).

Referring to FIG. 2, with continuing reference to FIG. 1, the foreground sight 34 and background sight 36 may include various geometries. The foreground sight 34 may include a substantially elliptical (e.g., circular) sight body 38. The background sight 36 may include a substantially cross-shaped sight body 40. In implementations, the sight body 38 of the foreground sight 34 may be situated in an opening 42. The opening 42 may be established along the proximal face 43 of the proximal portion 28. In implementations, the opening 42 may have a substantially elliptical (e.g., circular) shaped geometry. For the purposes of this disclosure, the terms “substantially” and “approximately mean ±10 percent of the stated value or relationship unless otherwise indicated.

The sight body 38 of the foreground sight 34 may have an opening 44. A perimeter of the opening 44 may substantially correspond to a geometry of a central portion 46 of the background sight 36 (e.g., FIG. 2). In implementations, the opening 44 may have a cross-shaped geometry. The central portion 46 of the background sight 36 may have a cross-shaped geometry. Other geometries of the opening 44 and central portion 46 may be utilized to indicate alignment between the foreground and background sights 34, 36, including a linear, T-shaped or complex profile. The opening 44 may have a profile dimensioned to substantially follow a profile of the central portion 46 of the background sight 36. The opening 44 may be alignable with the central portion 46 of the background sight 36. A substantially arc-shaped channel 60 may extend about a periphery of the foreground sight 34. Ends of the channel 60 may be established on opposite sides of a support (e.g., post) 45. In implementations, the foreground sight 34 may be cantilevered from the post 45, which may improve visibility around the periphery of the sight body 38. The channel 60 may extend at least 180 degrees, or more narrowly at least 315 degrees, about the periphery of the sight body 38. An outer periphery of the channel 60 may have a profile dimensioned to substantially follow a profile of an outer periphery of respective openings 47 distributed about the background sight 36 (see also FIGS. 4-5). In the implementation of FIGS. 4-5, the openings 47 may extend from a distal face 51 of the guide body 22.

The background sight 36 may include first and second alignment members 48, 50. The alignment members 48, 50 may intersect at a junction 52 to establish a substantially crossed shaped geometry. The junction 52 may be established at the central portion 46 of the background sight 36. The junction 52 may have a substantially elliptical (e.g., circular) geometry.

The alignment guide 20 may include one or more features to selectively permit projection of one or more (e.g., laser) beams of light along surface(s) of the background sight 36. The guide body 22 may include one or more alignment openings (e.g., channels). The channels may be dimensioned to facilitate oblique entry of one or more laser beams emitted from an adjacent imaging device. The alignment openings may have various geometries and arrangements. In implementations, the alignment openings may include a set of (e.g., elongated) channels along an exterior of the guide body 22. In implementations, the channels may include a first channel 54, a second channel 56, and/or a third channel 58. The channels 54, 56, 58 may be distributed about the axis X of the guide body 22. The first and third channels 54, 58 may be established on opposite sides of the foreground sight 34. The channels 54, 56 and/or 58 may be interconnected by the cavity 32. A width of the channels 54, 56, 58 may be substantially equal to a width of the alignment members 48, 50 of the background sight 36 (e.g., FIG. 2). A ratio between the width and a length of the respective channels 54, 56, 58 may be less than approximately 1:2, or more narrowly less than approximately 1:4 or approximately 1:10, which may facilitate relatively precise alignment relative to a (e.g., diffracted) laser beam.

The first, second, and/or third channels 54, 56, 58 may extend along respective reference planes REF1, REF2. In implementations, the reference planes REF1, REF2 may intersect adjacent to a center of the foreground sight 34 and/or may intersect adjacent to a center of the background sight 36 (e.g., FIG. 2). The reference planes REF1, REF2 may intersect at the foreground sight 34 and/or background sight 36 such that the channels 54, 56, 58 may establish respective pathways for one or more (e.g., a set of) laser beams projectable onto the background sight 36. The laser beams may be adapted to intersect at an optical center of a field of view of an associated imaging device. The first channel 54 may extend along the first reference plane REF1 between the first sidewall 24 and the cavity 32. The third channel 58 may extend along the first reference plane REF1 between the third sidewall 26 and the cavity 32. The channels 54, 58 may be substantially aligned with each other along the first reference plane REF1. The second channel 56 may extend along the second reference plane REF2 between the second sidewall 25 and the cavity 32. The first and second references planes REF1, REF2 may be substantially perpendicular or otherwise transverse to each other and may intersect the background sight 36. The first, second, and/or third channels 54, 56, 58 may extend from the proximal face 28 of the proximal portion 28. The arc shaped channel 60 may extend about the periphery of the foreground sight 34 to interconnect the first, second, and/or third channels 54, 56, 58 along the proximal face 43. The second channel 56 may be established between the sections 33 of the guide body 22. The first and third channels 54, 58 may be dimensioned to follow a wall of the respective sections 33. The sections 33 of the guide body 22 may serve to partially obstruct visibility of the background sight 36, including adjacent to the channels 54, 56, 58.

The first and second reference planes REF1, REF2 may intersect along the opening 44 of the foreground sight 34 and/or the central portion 46 of the background sight 36 (e.g., FIG. 2). The first and/or second reference planes REF1, REF2 may intersect along the axis X of the guide body 22. The axis X of the guide body 22 may extend along first and/or second reference planes REF1, REF2. The reference planes REF1, REF2 may intersect along a length of the axis X. The first and/or second reference planes REF1, REF2 may follow the respective first and second alignment members 48, 50 and may intersect the junction 52 and/or another position along the central portion 46 of the background sight 36.

Referring to FIGS. 3-5, with continuing reference to FIGS. 1-2, the guide body 22 may include one or more openings (e.g., viewing windows) 49. The viewing windows 49 may be established along the respective sidewalls 24, 25, 26. The viewing windows 49 may be dimensioned to interconnect the cavity 32 and an exterior of the guide body 22. The viewing windows 49 may be established adjacent to the background sight 36 and may be dimensioned to permit visibility of the background sight 36 from an oblique angle relative to the axis X of the guide body 22. The channels 54, 56, 58 may be dimensioned to extend proximally from the respective viewing windows 49 to the proximal face 43 of the guide body 22. In implementations, a maximum width of the viewing window 49 may be greater than a maximum width of the respective channel 54, 56, 58 (e.g., FIG. 3). The viewing windows 49 may facilitate projecting and/or visually aligning the laser beams along the background sight 36, whereas the channels 54, 56, 58 may assist in aligning the axis X of the guide body 22 relative to a field of view of an imaging device associated with the laser beams.

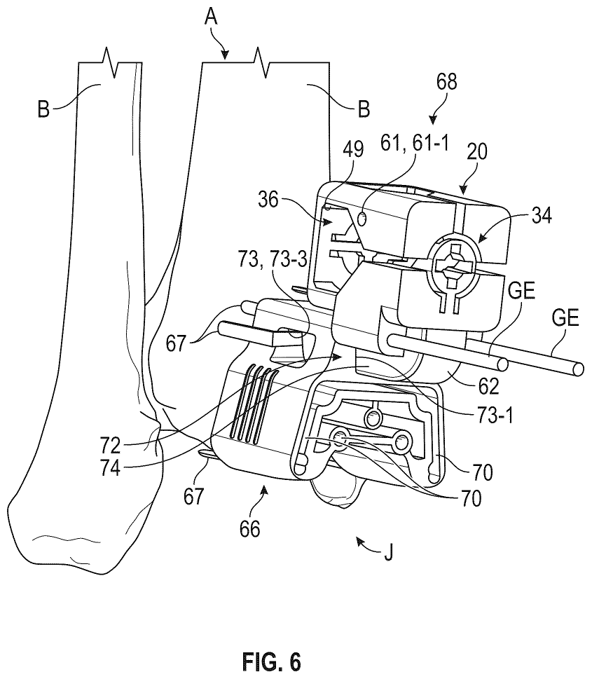

The guide body 22 may include one or more alignment features 61 (e.g., FIG. 3). The alignment feature 61 may be useful in confirming alignment of the alignment guide 20 in another view. In implementations, the alignment feature 61 may be an aperture (e.g., passage). An axis XA of the alignment feature 61 may be substantially perpendicular to the axis X of the guide body 22. A cross section of the alignment feature 61 may be in the shape of a (e.g., perfect) circle machined or otherwise formed within precise tolerance(s). The guide body 22 may include a pair of alignment features 61-1, 61-2 which may be visually aligned with each other to confirm or otherwise establish an orientation of the alignment guide 20 (e.g., FIGS. 6 and 8). In other implementations, the alignment feature(s) 61 may be omitted.

The guide body 22 may be securable to a surgical instrument, such as an instrument guide including any of the instrument guides disclosed herein. The guide 20 may include various structures for fixedly attaching or otherwise securing the guide 20 to the surgical instrument. The alignment guide 20 may include a mount 62 for securing the alignment guide 20 to the surgical instrument. The mount 62 may extend outwardly from the guide body 22. In implementations, the mount 62 may have a generally L-shaped geometry (e.g., FIG. 3). The mount 62 may include a protrusion that may be dimensioned to mate with a receptacle of the surgical instrument. In other implementations, the guide body 22 may include a receptacle dimensioned to mate with a protrusion of the surgical instrument.

The alignment guide 20 may include one or more guide passages 64 (e.g., FIGS. 2 and 4-5). The guide passage 64 may have various geometries, such as a substantially elliptical (e.g., circular) geometry or a slot. Each guide passage 64 may be dimensioned to receive a surgical device. The surgical device may include a cutting instrument or guide element GE (shown in dashed lines in FIG. 2). Various cutting instruments may include a drill, reamer or saw blade suitable for removing bone or other tissue. Various guide elements may be utilized, such as a Kirshner (K-wire) insertable in bone. The guide passages 64 may be established in the guide body 62. The guide passages 64 may include first and second guide passages 64 that may extend along respective passage axes PX (e.g., FIGS. 2 and 5). The passage axes PX may be substantially parallel to each other. The guide element GE may be positioned along the respective passage axis PX. The alignment guide 20 may be movable along the guide element(s) GE to position the guide 20 relative to the anatomy.

Various techniques may be utilized to establish the alignment guide 20. The guide body 22 and/or other portions of the alignment guide 20 may include various metallic materials, such as a surgical grade steel or titanium, and/or non-metallic materials such as a biocompatible polymer. In implementations, the guide body 22 may include a radiopaque material including any metallic materials disclosed herein. The guide body 22 may be monolithic or may include one or more components fixedly attached or otherwise secured to each other. In implementations, the guide body 22 may be radiopaque such that visibility of the background sight 36 may be partially obstructed by portions of the guide body 22 adjacent to the channels 54, 56 and/or 58 and/or viewing windows 49. The techniques disclosed herein may be used to improve accuracy and alignment between the features of the foreground sight 34 and/or background sight 36 relative to laser beam(s) and/or an optical center of the field of view of an associated camera.

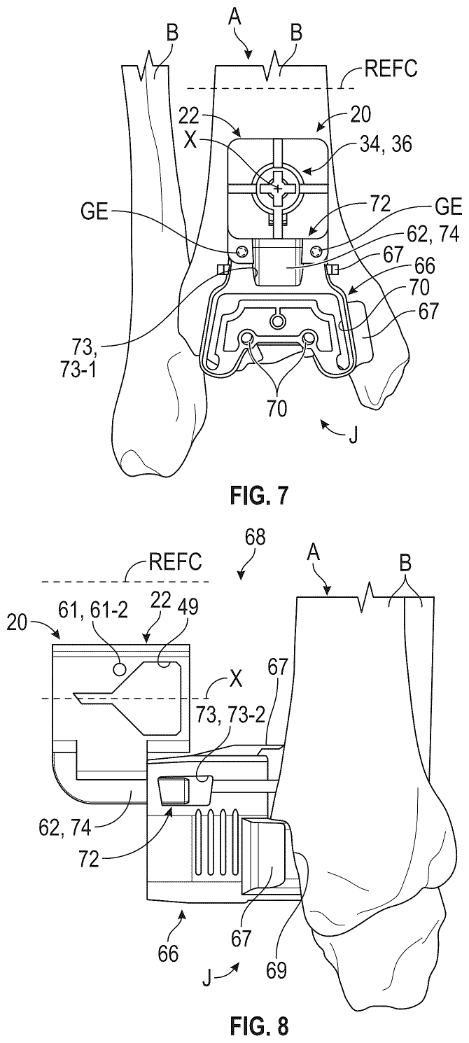

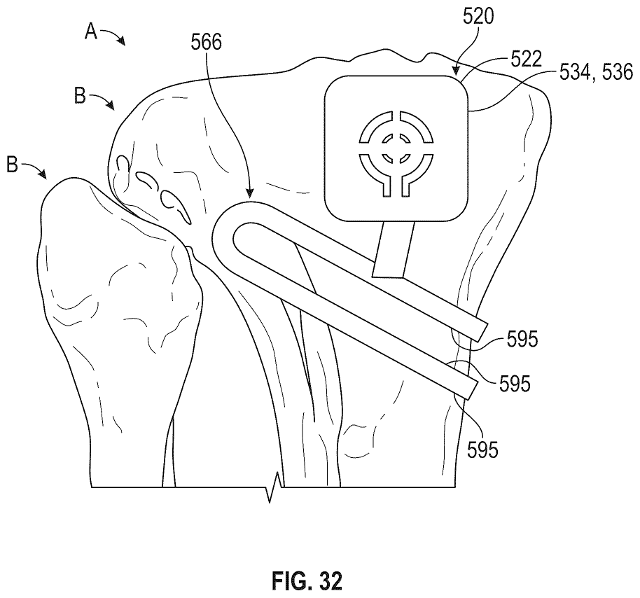

Referring to FIGS. 6-8, with continuing reference to FIGS. 1-3, the alignment guide 20 may be utilized to position an anatomy A of a patient and/or an instrument guide 66 relative to a field of view of an imaging device. The anatomy A may include one or more bones B and/or joints J of a patient, including any of the bones and joints disclosed herein. The alignment guide 20 may be a separate component or may be integrally formed with the instrument guide 66 (e.g., instrument guide 520 of FIG. 32). The alignment guide 20 and the instrument guide 66 may be provided in a surgical kit. The alignment guide 20 and instrument guide 66 may be configured to establish an assembly 68. The foreground sight 34 and the background sight 36 may be visually alignable with each other to indicate an orientation of the instrument guide 66 relative to a line of sight of the surgeon or clinical user and/or a field of view of an imaging device (e.g., FIG. 7).

The instrument guide 66 may include on or more apertures 70 (e.g., FIGS. 6-7) dimensioned to guide a surgical device, including any of the surgical devices disclosed herein. The apertures 70 may include one or more passages and/or slots to guide various surgical devices such as a drill, guide element, saw blade, etc. The instrument guide 66 may be adapted to contact an anatomical surface of the anatomy A based on a surgical (e.g., preoperative) plan associated with a patient. In implementations, the instrument guide 66 may include one or more locating members 67. The locating members 67 may include one or more patient-specific dimensions and/or patient-specific surfaces 69 (e.g., FIG. 8) to establish a pre-selected position and orientation of the instrument guide 66 relative to an anatomical surface of the anatomy A. The patient-specific surface 69 may have a contour that may be a substantial negative of a contour of a respective surface of the anatomy A, including an articular and/or non-articular surface of a bone. In other implementations, the locating member(s) 67 and/or another portion of the alignment guide 66 may be configurable to establish the position and orientation of the alignment guide 66 relative to the anatomy A based on one or more settings, which may be specified in the surgical plan.

The alignment guide 20 may be fixedly attached or (e.g., releasably) securable to the instrument guide 66 at one or more interfaces 72. Each interface 72 may be dimensioned to establish a preselected orientation of the alignment guide 20 relative to the instrument guide 66. The interface 72 may include a receptacle 73 dimensioned to mate with a protrusion 74 to orient the alignment guide 20 at a preselected orientation relative to the instrument guide 66. The protrusion 74 may be established by the mount 62 of the alignment guide 20. The receptacle 73 may be established by a body of the instrument guide 66. In other implementations, the instrument guide 66 may include the protrusion 74 and the alignment guide 20 may include the receptacle 73. The protrusion 74 and receptacle 73 may be dimensioned to establish an interference fit for securing the alignment guide 20 and instrument guide 66 to each other.

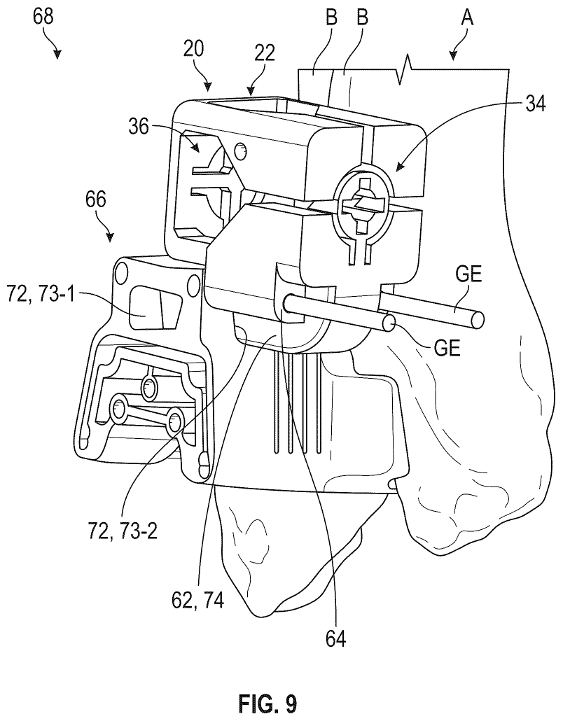

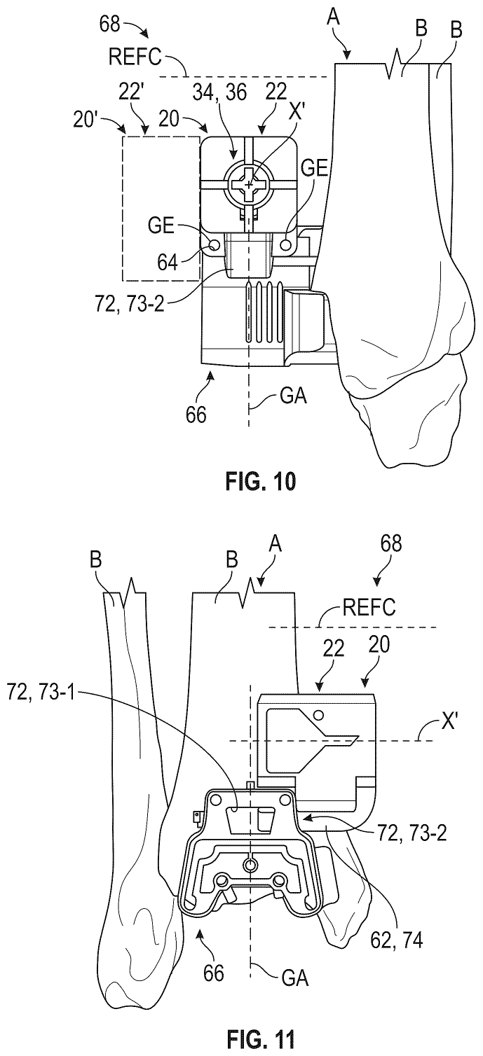

In the implementation of FIGS. 6-8 and 9-11, the instrument guide 66 may include a set of interfaces 72. The interfaces 72 may be associated with a set of receptacles 73-1, 73-2, and/or 73-3 which may be established by the instrument guide 66. The receptacles 73-1, 73-2, 73-3 may be established at different orientations relative to each other. In implementations, the second and third receptacles 73-2, 73-3 may be on opposite sides of the instrument guide 66. The receptacle 73-1 may be established between the receptacles 73-2, 73-3. In implementations, the receptacles 73-1, 73-2, 73-3 may be established at approximately 90 degree increments relative to an axis GA of the instrument guide 66 (e.g., FIGS. 10-11). The guide body 22 of the alignment guide 20 may be situated a first orientation relative to the instrument guide 66 at the first interface 73-1 (e.g., FIGS. 6-8). The guide body 22 may be situated a second orientation relative to the instrument guide 66 at the second interface 73-2 (e.g., FIGS. 9-11). The guide body 22 may be situated at a third orientation relative to the instrument guide 66 at the third interface 73-3 (e.g., opposite the arrangement of FIGS. 9-11). One or more guide elements GE may be insertable through the guide passages 64 and respective passages in the instrument guide 66 to align the alignment guide 20 and instrument guide 66 to relative to each other (e.g., FIGS. 6 and 9-10). In implementations, an alignment guide may include two sets of foreground and background sights and associated features of the guide body (e.g., a combination of the guide 20 of FIG. 10 and the guide 20 of FIG. 8 shown in dashed lines at 20′).

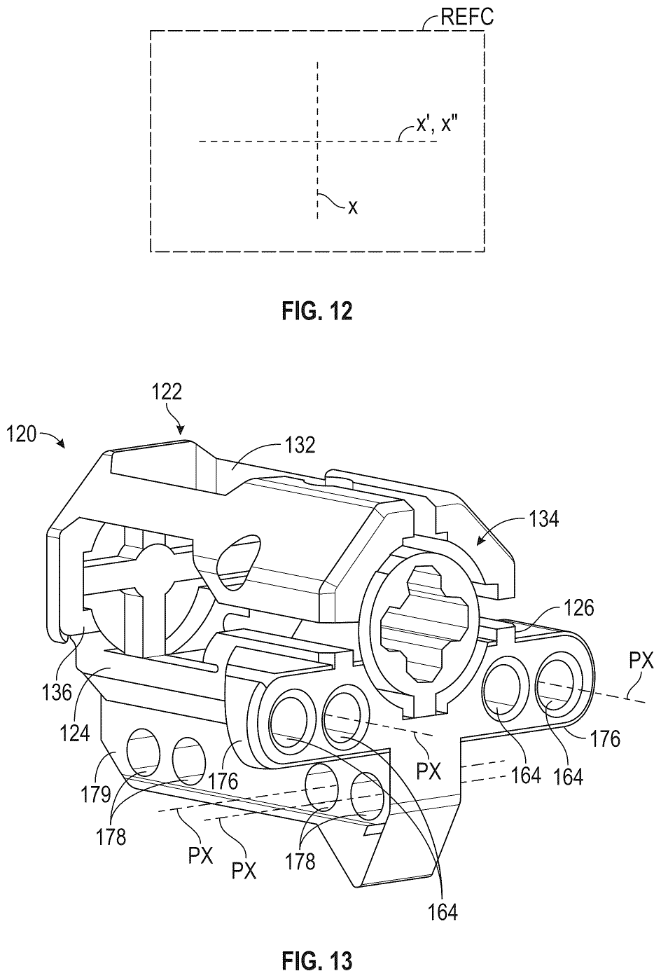

In implementations, the interfaces 72 may be established such that a position of the axis X of the guide body 22 associated with the first orientation (e.g. FIG. 7) may be substantially perpendicular to a position of the axis X′ associated with the second orientation (e.g., FIG. 10) when projected onto a common reference plane REFC. In implementations, the receptacle 73-3 may be established in the instrument guide 66 such that the axis X″ of the guide body 22 (FIG. 12) may be substantially co-linear with, or otherwise parallel to, the axis X′ associated with the second orientation of the alignment guide 20. FIG. 12 discloses an arrangement of the axes X, X′, X″ relative to the reference plane REFC.

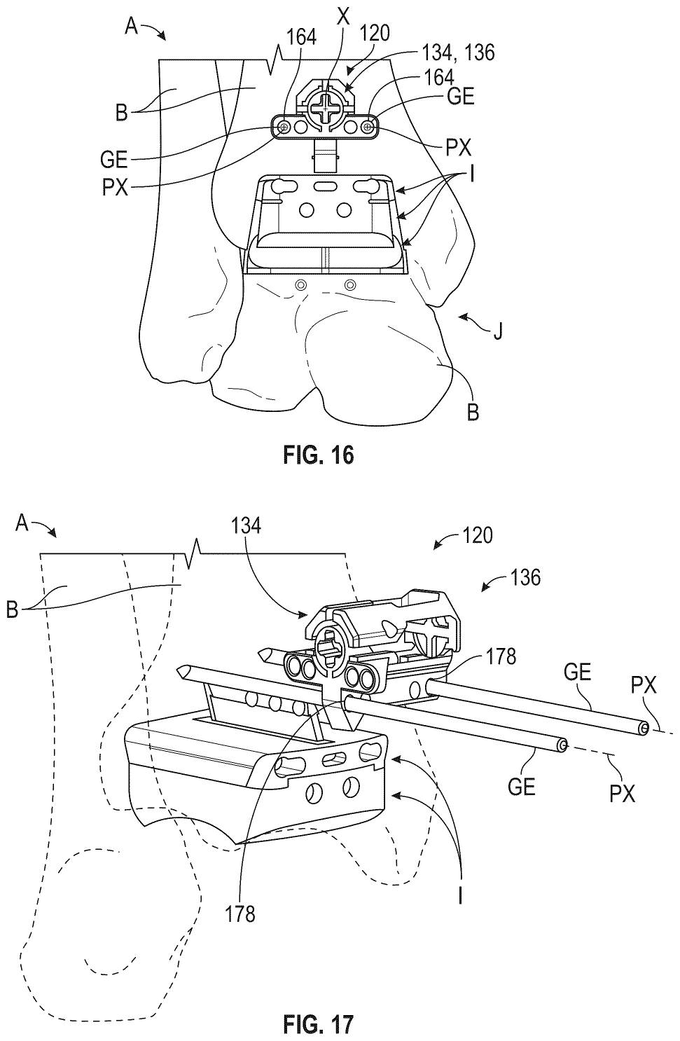

FIGS. 13-18 disclose an alignment guide 120 according to another implementation. In this disclosure, like reference numerals designate like elements where appropriate and reference numerals with the addition of one-hundred or multiples thereof designate modified elements that are understood to incorporate the same features and benefits of the corresponding original elements. The alignment guide 120 may be securable to one or more surgical devices, which may be positioned relative to the anatomy of a patient.

Referring to FIG. 13, the alignment guide 120 may include one or more guide passages 164 established in a guide body 122. The guide passages 164 may be established in one or more flanges 176. The flanges 176 may be dimensioned to extend from the guide body 122. In implementations, the alignment guide 120 may include a pair of flanges 176 that may extend from respective sidewalls 124, 126 on opposite sides of the guide body 122. In implementations, the alignment guide 120 may be a first set of guide passages 164. The alignment guide 120 may include a second set of guide passages 178. The guide passages 178 may be established in a flange 179, which may extend outwardly from the guide body 122. In implementations, the flanges 176 may be extend outwardly from, or may otherwise be established on, opposite sides of the flange 179. The guide passages 164, 178 may be dimensioned to extend along respective passage axes PX. In implementations, the passage axes PX of the guide passages 164 may be substantially parallel to each other. The passage axes PX of the guide passages 178 may be substantially parallel to each other. The passage axes PX associated with the guide passages 164 may be substantially perpendicular or otherwise transverse to the passage axes PX associated with the guide passages 178 such that the alignment guide 120 may be situated at different orientations relative to (e.g., common) guide element(s) GE positioned in the anatomy A.

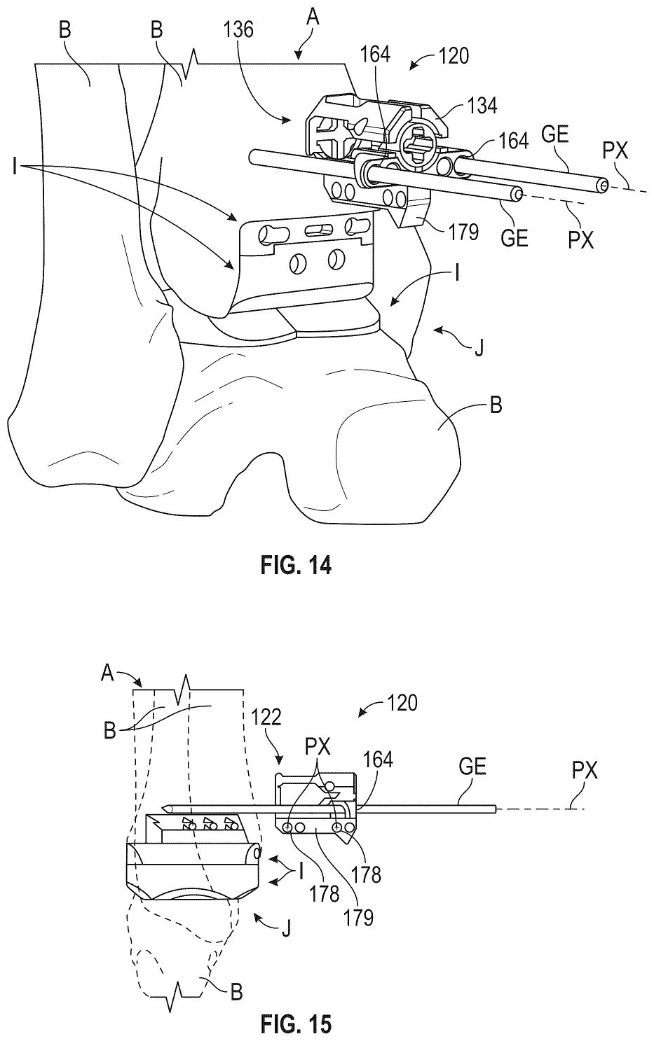

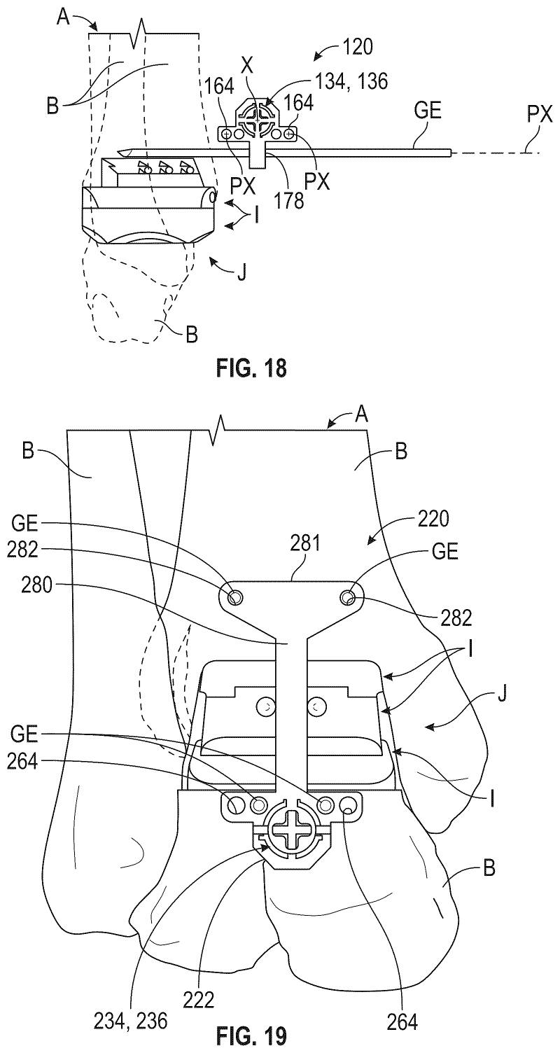

FIGS. 14-16 disclose the alignment guide 120 situated at a first orientation relative to the anatomy A. The alignment guide 120 may be secured relative to one or more guide elements GE, which may be inserted or otherwise positioned in the anatomy A. FIGS. 17-18 disclose the alignment guide 120 situated at a second orientation relative to the anatomy A, which may be substantially perpendicular to, or otherwise differ from, the first orientation. The guide elements GE of FIG. 14-16 and FIGS. 17-18 may be positioned at the same locations and trajectories relative to the anatomy A. In the implementation of FIGS. 17-18, the alignment guide 120 may be oriented in a position substantially perpendicular to the position associated with the alignment guide 120 of FIGS. 14-16. The guide elements GE may be adapted to position a surgical device relative to the anatomy A, including any of the instrument guides and other surgical devices disclosed herein. The guide elements GE may be utilized for implantation of a prothesis including one or more implant components I. The implant component(s) I may include an articular surface dimensioned to mate with an articular surface of an adjacent bone or implant component. In implementations, the alignment guide 120 may be adapted to mate with, or may be otherwise securable to, the implant component(s) I.

FIG. 19 discloses an alignment guide 220 according to another implementation. The guide 220 may include a guide body 222. One or more guide passages 264 may be established in the guide body 222. The alignment guide 220 may include a bridge member 280. The bridge member 280 may be dimensioned to interconnect the guide body 222 and a locating member 281. The guide body 222, bridge member 280 and locating member 281 may be integrally formed or may be separate components fixedly attached or otherwise secured to each other.

The bridge member 280 may be dimensioned to span across and interconnect adjacent bones B of a joint J of the anatomy A, including any of the joints and associated bones disclosed herein. In the implementation of FIG. 19, the bones B may include a tibia and a talus of an ankle joint of the patient.

One or more guide passages 282 may be established in the locating member 281. The guide passages 264 and/or guide passages 282 may be dimensioned to receive one or more guide elements GE. The alignment guide 220 may be adapted to fix a position of the adjacent bones B relative to each other in response to positioning the guide elements GE in the respective guide passages 264, 282. One or more implant components I may be positioned relative to the anatomy A. Fixing the adjacent bones B may improve alignment during imaging of the anatomy A and/or implant component(s) I. The captured images may be utilized to determine whether the implant component(s) I may be positioned relative to the anatomy A according to a surgical plan associated with the patient. The alignment guide 220 may be useful in (e.g., simultaneously) verifying the position and trajectory of the guide elements GE relative to the bones B prior to removing portion(s) of the bone B and/or placement of the implant component(s) I.

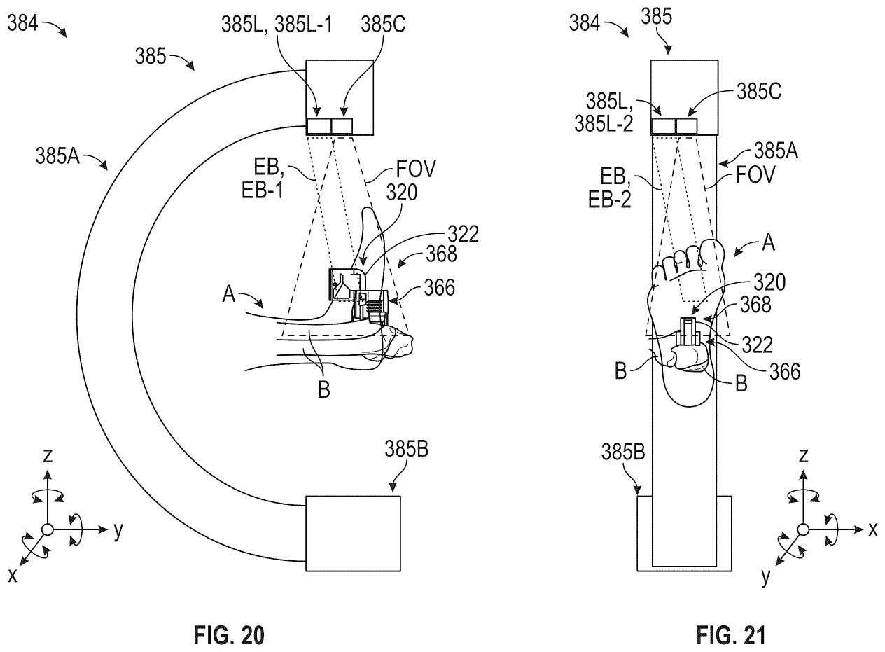

FIGS. 20-21 disclose a system 384 for surgical imaging according to an implementation. The system 384 may include an imaging device 385. Various imaging devices may be utilized, such as a fluoroscopic camera. In implementations, the imaging device 385 may be a C-Arm machine operable to perform fluoroscopic imaging during orthopaedic and other surgical procedures. The imaging device 385 may include a fluoroscopic camera 385C and one or more lasers 385L. The imaging device 385 may include an arm 385A secured to a base 385B. The arm 385A may be adapted to carry or otherwise situate the camera 385C and/or lasers 385L relative to the anatomy A of a patient. The arm 385A may be movable along, about and/or otherwise relative to one or more axes X, Y, Z. The camera 385C and/or lasers 385L may be carried by the arm 385A. The arm 385A may be movable to set a position of the camera 385C and/or lasers 385L relative to the anatomy A. The camera 385C may be associated with a field of view FOV (shown in dashed lines).

The lasers 385L may be operable to emit respective laser beams EB along respective paths (shown in dashed lines). Each beam EB may be diffracted (e.g., split) such that a set of the split beams may extend along a plane. In implementations, the beams EB may be oblique to the field of view FOV of the camera 385C such that the beams EB may intersect each other adjacent to an optical center OC of the field of view FOV (e.g., FIG. 22).

The lasers 385L may include a first laser 385L-1 and/or a second laser 385L-2. The lasers 385L-1, 385L-2 may be distributed about the camera 385C. The laser 385L-1, 385L-2 may be spaced apart from each other relative to one or more of the axes X, Y, Z. The lasers 385L-1, 385L-2 may be associated with respective beams EB-1, EB-2. The laser beams EB-1, EB-2 may intersect each other to establish a crosshair. In implementations, the laser beams EB-1, EB-2 may intersect each other at, or otherwise adjacent to, the optical center OC of the field of view FOV of the camera 385C (e.g., FIG. 22).

The system may include an alignment guide 320. The alignment guide 320 may include any of the alignment guides disclosed herein. The alignment guide 320 may include a guide body 322. The guide body 322 and/or other portions of the alignment guide 320 may incorporate any of the materials disclosed herein, such as a radiopaque material.

Referring to FIG. 22, with continuing reference to FIGS. 20-21, the alignment guide 320 may include a foreground sight 334 and/or background sight 336. The foreground and background sights 334, 336 may be visually alignable with each other to indicate an orientation of the guide body 322 relative to the field of view FOV of the camera 385C.

The alignment guide 320 may include a set of channels along an exterior of the guide body 322. In implementations, the guide body 322 may include first, second, and/or third channels 354, 356, 358. The channels 354, 356, 358 may extend along respective reference planes REF1, REF2 that may intersect the foreground sight 334 and/or background sight 336 (see also FIG. 2). The background sight 336 may serve as a target. The channels 354, 356, 358 may be dimensioned to permit a projection of the respective laser beams EB-1, EB-2 onto the background sight 336. The laser beams EB-1, EB-2 may intersect the background sight 336 at a position aligned with a visual path between the foreground and background sights 334, 336, which may extend along an axis X of the alignment guide 320.

The system 384 may include an instrument guide 366. The instrument guide 366 may include any of the instrument guides disclosed herein. The instrument guide 366 may be adapted to contact an anatomical surface of the anatomy A of a patient based on a surgical plan. The alignment guide 320 and instrument guide 366 may be configurable to establish an assembly 368. The alignment guide 320 may be securable to the instrument guide 366 to indicate an orientation of the instrument guide 366 relative to the field of view FOV of the camera 385C. The alignment guide 320 may be secured to the instrument guide 366 at one or more interfaces 372. An axis X of the alignment guide 320 may be arranged at a substantially perpendicular angle relative to the field of view FOV.

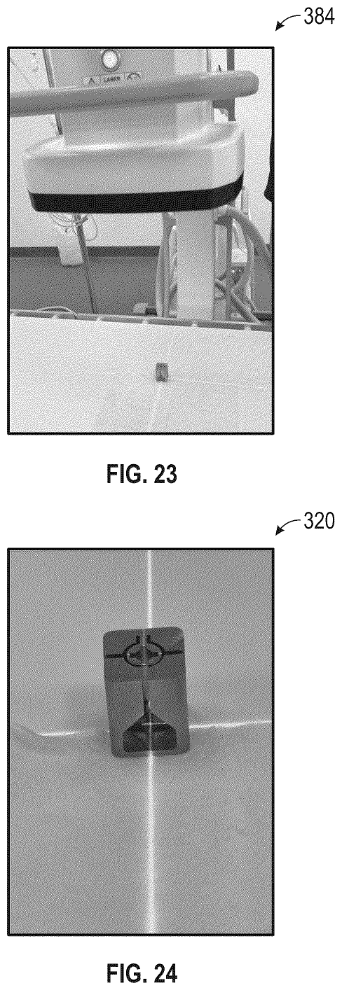

FIG. 23 discloses an image of the implementation of the system 384 including an imaging device. The imaging device may include a fluoroscopic camera and a set of laser that may emit laser beams onto a surface of an alignment guide, such as the alignment guide 320. FIG. 24 discloses an image of the laser beams intersecting a surface of the background sight of the alignment guide. The surgeon or clinical user may substantially align the alignment guide relative to an optical center of the camera prior to capturing one or more images of the anatomy with the camera.

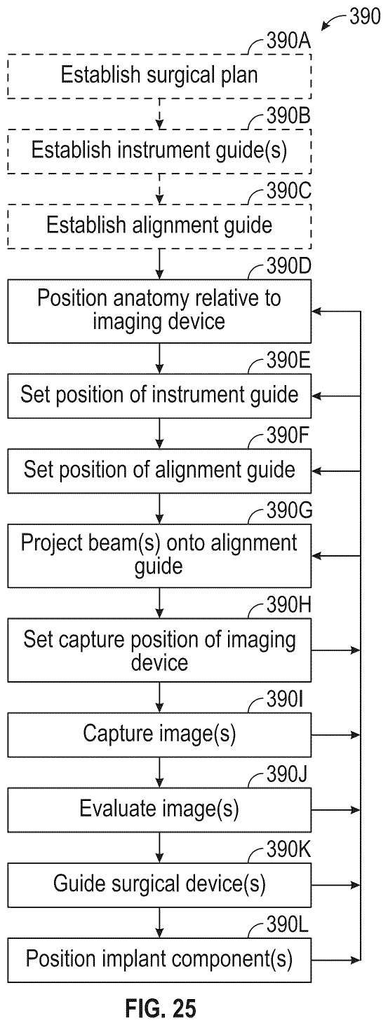

FIG. 25 discloses a method for a surgical imaging in a flowchart 390 according to an implementation. The method 390 may be utilized with any of the systems, alignment guides, imaging devices and/or surgical devices disclosed herein. The method 390 may be utilized to capture one or more images of an anatomy of a patient during an orthopaedic and other surgical procedure. The anatomy may include various bones and joints, including any of the bones and associated joints disclosed herein. The method 390 may be useful in performing fluoroscopic imaging of the patient anatomy. Fewer or additional steps than are recited below could be performed within the scope of this disclosure, and the recited order of steps is not intended to limit this disclosure. Reference is made to the system 384.

Referring to FIGS. 20-22, with continuing reference to FIG. 25, a surgical plan may be established for a patient at block 390A. The surgical plan may be established preoperatively and/or intraoperatively. The plan may include one or more parameters, such as a (e.g., patient-specific) position and/or orientation of the alignment guide 320 and/or instrument guide 366 relative to the anatomy A. The parameters may include one or more settings for configuring the instrument guide 366 to implement the surgical plan. The plan may include values of one or more dimensions associated with a geometry of the alignment guide 320, including any of the dimensions disclosed herein.

At block 390B, one or more instrument guides may be established. The instrument guides may include any of the instrument guides disclosed herein, such as the instrument guide 366. The instrument guide 366 may be established based on the surgical plan established at block 390A. The instrument guide 320 may incorporate any of the materials disclosed herein.

At block 390C, an alignment guide may be established. The alignment guide may include any of the alignment guides disclosed herein, such as the alignment guide 320. The alignment guide 320 may incorporate any of the materials disclosed herein, such as a radiopaque material. The alignment guide 320 may include a foreground sight 334 and a background sight 336 which may be opposed to each other. The alignment guide 320 may include a cavity 332 bounded by the foreground and background sights 334, 336. In implementations, the foreground sight 334 may have a generally ring-shaped geometry and may be dimensioned to silhouette a generally cross-shaped geometry of the background sight 336 (e.g., FIG. 22), or vice versa. Visibility of the background sight 336 may be partially obstructed by the guide body 322. The alignment guide 320 may include one or more openings to permit visibility of the background sight 336 from an exterior of the guide body 322. In implementations, the alignment guide 320 may include a set of elongated channels, such as the channels 354, 356 and/or 358 (FIG. 22). The channels 354, 356, 358 may be distributed along an exterior of the guide body 322 and/or another portion of the alignment guide 320.

At block 390D, the anatomy A of the patient may be positioned relative to the imaging device 385. The imaging device 385 may include a fluoroscopic camera 385C and one or more lasers 385L. In implementations, the imaging device 385 may include a set of lasers, such as the first and second lasers 385L-1, 385L-2. The lasers 385L-1, 385L-2 may be operable to emit laser beams EB-1, EB-2 along respective paths, which may be oblique to the field of view FOV of the camera 385C. In implementations, the camera 385C and/or lasers 385L may be moved relative to the anatomy A or vice versa, such that the anatomy A may be within the field of view FOV of the camera 385C.

At block 390E, a position of the instrument guide 366 may be set along or otherwise relative to the anatomy A. Various techniques may be utilized to set the position of the instrument guide 366, including any of the techniques disclosed herein. The position of the instrument guide 366 may be set at a patient-specific position and/or orientation, which may be specified in the surgical plan. The instrument guide 366 may be adapted to contact the anatomy A at a patient-specific position and/or orientation. In implementations, the instrument guide 366 may include one or more locating members 367. The locating members 367 may be dimensioned according to any of the techniques disclosed herein. The locating members 367 may include one or more patient-specific dimensions and/or patient-specific surfaces (e.g., surface 69 of FIG. 8). In other implementations, the locating members 367 and/or other portions of the instrument guide 366 may be configured to establish the patient-specific position and/or orientation based on the surgical plan. In implementations, the instrument guide 366 may be omitted.

At block 390F, a position of the alignment guide 320 may be set relative to the anatomy A. Various techniques may be utilized to set the position of the alignment guide 320, including any of the techniques disclosed herein. In implementations, the alignment guide 320 may be set relative to one or more guide elements (e.g., FIGS. 6 and 14-19). Setting the position of the alignment guide 320 may include positioning the alignment guide 320 on one or more guide elements GE positioned in the anatomy A. The location and trajectory of the guide element(s) GE relative to the anatomy A may be specified in the surgical plan. Setting the position of the alignment guide 320 may include fixedly attaching or otherwise securing the alignment guide 320 to the instrument guide 366. Setting the position of the alignment guide 320 may include securing the alignment guide 320 at a (e.g., first or selected) interface 372 of the instrument guide 366.

Setting the position of instrument guide 366 at block 390E and/or setting the position of the alignment guide 320 at block 390F may include positioning the instrument guide 366 and/or alignment guide 320 within the field of view FOV of the camera 385C. The foreground sight 334 and/or background sight 336 may be positioned at or otherwise adjacent to the optical center OC of the camera 385C.

At block 390G, one or more light (e.g., laser) beams may be projected onto the alignment guide 320. In implementations, one or more laser beams EB (e.g., EB-1, EB-2) may be projected (e.g., emitted) from the respective lasers 385L. The laser beams EB-1, EB-2 may be projected along respective paths such that the laser beams EB-1, EB-2 may intersect each other at, or otherwise adjacent to, the optical center OC of the field of view FOV of the camera 385C (e.g., FIG. 22, see also FIGS. 23-24). In implementations, block 390G and associated features of the alignment guide 320 may be omitted.

At block 390H, a capture position of the imaging device 385 may be set relative to the alignment guide 320. Setting the capture position may include establishing foreground and background alignment between the sights 334, 336 relative to the field of view FOV of the camera 385C. The capture position may be set such that the foreground sight 334 and background sight 336 may be substantially aligned with each other relative to the camera field of view FOV and/or such that the laser beams EB-1, EB-2 may intersect each other along the background sight 336. Setting the capture position may include projecting the laser beams EB-1, EB-2 through the respective channels 354, 356, 358 onto the background sight 336.

The channels 354, 356, 358 may extend along respective reference planes REF1, REF2 that may be substantially perpendicular or otherwise transverse to each other. The reference planes REF1, REF2 may intersect the foreground sight 334 and/or background sight 336. Block 390H may include simultaneously projecting the laser beam EB-2 through both channels 354, 358, which may be established on opposite sides of the foreground sight 334.

Setting the capture position may include projecting the laser beams EB-1, EB-2 along respective alignment members 348, 350 of the background sight 336 such that the laser beams EB-1, EB-2 may intersect each other at a junction 352 between the alignment members 348, 350 (e.g., FIG. 22). Setting the capture position may include repeating one or more iterations of blocks 390D, 390E, 390F, and/or 390G until sufficient alignment may be achieved. Setting the capture position may occur such that the axis X of the alignment guide 320 may be substantially perpendicular to the field of view FOV and/or may be substantially aligned with the optical center OC of the camera 385C (e.g., FIG. 22).

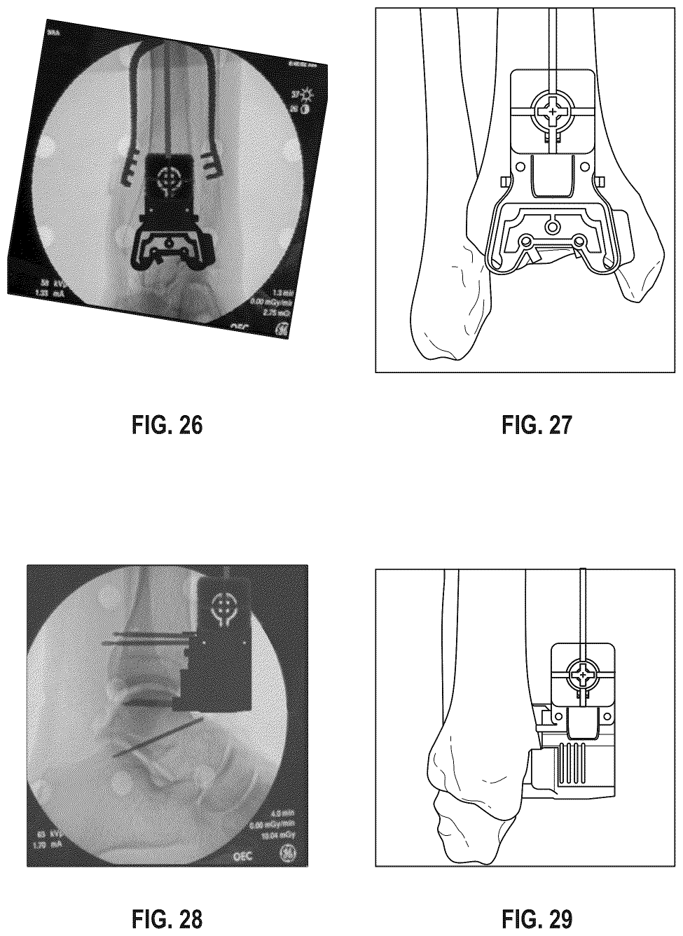

At block 390I, one or more images may be captured using the camera 385C at the capture position. The image(s) may include portions of the anatomy A, alignment guide 320 and/or instrument guide 366. FIG. 26 depicts an image of a first (e.g., coronal) view of an implementation of the alignment guide 320 and instrument guide 366 relative to the anatomy A. FIG. 28 depicts an image of a second (e.g., sagittal) view of an implementation of the alignment guide 320 and instrument guide 366 relative to the anatomy A.

The method 390 may include securing the alignment guide 320 at another (e.g., second) interface 372 of the instrument guide 366, and then repeating one or more iterations of positioning the anatomy A at block 390D, projecting the beams EB at block 390G, setting the capture position at block 390H and/or capturing one or more images at block 390I. The captured images associated with the alignment guide 320 at the first and second interfaces 372 (e.g., FIGS. 7 and 10) may correspond to different anatomical planes of the anatomy. The anatomical planes may include a coronal (e.g., frontal) plane (e.g., FIG. 26), sagittal (e.g., longitudinal) plane (e.g., FIG. 28) and/or transverse (e.g., axial) plane of the anatomy.

At block 390J, the captured image(s) may be evaluated. Each captured image may be compared to a virtual depiction of the alignment guide 320 relative to the anatomy A associated with the surgical plan for the patient. FIG. 27 discloses a virtual depiction of a coronal view of the alignment guide 320 and an implementation of the instrument guide 366 relative to the anatomy A, which may be compared to the image of FIG. 26. FIG. 29 discloses a virtual depiction of a sagittal view of an implementation of the alignment guide 320 and instrument guide 366 relative to the anatomy A, which may be compared to the image of FIG. 28. An alignment device (e.g., extramedullary rod) 394 may be fixedly attached or otherwise secured to the alignment guide 320 (shown in dashed lines in FIG. 22, see also FIGS. 26-29). The extramedullary rod 394 may be oriented relative to a (e.g., longitudinal) axis of the adjacent bone. Block 390J may include visually comparing an orientation of the extramedullary rod 394 in the captured image to an orientation of the extramedullary rod 394 in the virtual depiction.

At block 390K, one or more surgical devices may be guided relative to the anatomy A. Block 390K may include guiding, using the instrument guide 366, at least one or more surgical devices relative to the anatomy A, which may occur subsequent to the capturing the image(s) at block 390I. The surgical device may include any of the surgical devices disclosed herein.

At block 390L, one or more implant components may be positioned relative to the anatomy A (e.g., FIGS. 14-19). In implementations, the implant component(s) may be positioned relative to the anatomy A using the guide element(s), which may occur subsequent to capturing the image(s) at block 390I. The alignment guide 320 may be positioned along the guide element(s) to capture one or more images prior to, during and/or subsequent to positioning the implant component(s).

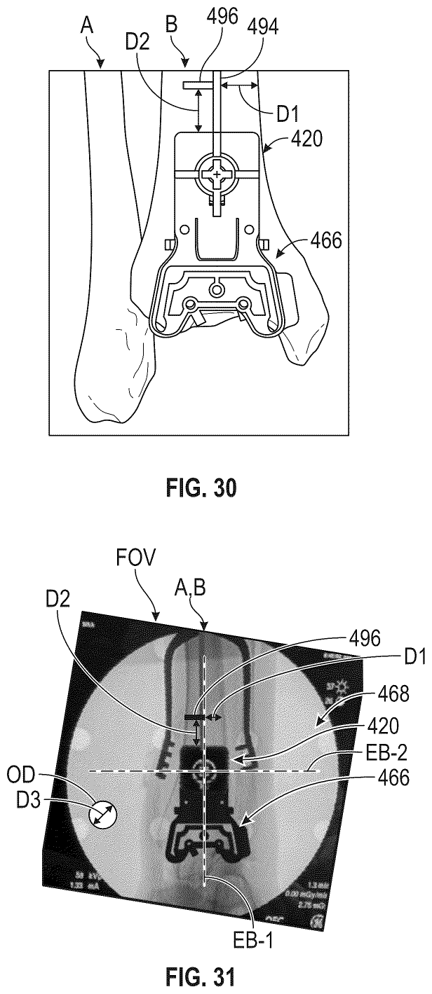

Referring to FIGS. 30-31, with continuing reference to FIGS. 20-21 and 25, one or more dimensional features may be utilized to determine positioning of an alignment guide relative to a field of view FOV of an imaging device. FIG. 30 discloses a virtual depiction of an assembly 468 including an alignment guide 420 and instrument guide 466 positioned relative to an anatomy A and/or within the field of view FOV based on a surgical plan for a patient. FIG. 31 discloses an interoperative image of the assembly 468 including the alignment guide 420 and the instrument guide 466 positioned relative to the anatomy A of the patient based on the surgical plan. Setting the capture position at block 390H may include comparing a value of dimension(s) associated with the alignment guide 420 and the anatomy A in the captured image to a value of the dimension(s) in the virtual depiction of the alignment guide 420 relative to the anatomy A.

The alignment guide 420 may be associated with a known object reference 496. The known object reference 496 may be associated with a known distance D2 from the alignment guide 420. The known object reference 496 may have various geometries. In implementations, the known object reference 496 may be an elongated member (e.g., reference pin). The dimension D2 may extend substantially perpendicular to an axis of the known object reference 496 and/or a surface of the alignment guide 420. In implementations, the known object reference 496 may be associated with an alignment device (e.g., extramedullary rod) 494. The extramedullary rod 494 may be oriented relative to a (e.g., longitudinal) axis of the adjacent bone B.

One or more dimensions may be determined between the known object reference 496 and anatomy A. A dimension D1 may be determined between the known object reference 496 and a contour of the bone B. The dimension D1 may extend substantially parallel to an axis of the known object reference 496. In other implementations, the dimension D1 may be determined between the known reference object 496 and another portion of the anatomy, such as a contour of the soft tissue (e.g., skin) adjacent to the known reference object 496. Values of the dimensions D1, D2 may be specified in the surgical plan. Evaluating the captured image(s) at block 390J may include determining values of the dimensions D1, D2 in the captured image and comparing the determined values to the associated values of the dimensions D1, D2 specified in the surgical plan. The captured image may be associated with a known object dimension OD. The known object dimension OD may be associated with a feature of the alignment guide 420 or a separate object (e.g., calibration block). The known object dimension OD may be associated with a known dimension D3. A value of the known dimension D3 in the captured image may be determined and may be utilized to scale the determined values of the dimensions D1, D2. A deviation between the values of the dimensions D1 and/or D2 (and/or a ratio of D1: D2) exceeding a preselected threshold (e.g., 5 percent) with respect to the values specified in the surgical plan may indicate misalignment of the alignment guide 420 and anatomy A relative to the field of view FOV of the camera. Misalignment may occur due to rotation of a limb of the patient relative to the field of view FOV. The surgeon or clinical user may adjust a position and/or orientation of the anatomy A and camera relative to each other to reduce the deviation and achieve a suitable alignment utilizing any of the techniques disclosed herein. The dimensions may be compared to verify that the alignment guide 420 and/or associated instrument guide may be in the correct position as specified in the surgical plan.

FIG. 32 discloses an alignment guide 520 according to another implementation. The alignment guide 520 may be integrally formed or otherwise secured to an instrument guide 566. The instrument guide 566 may include one or more features dimensioned to guide another surgical instrument. In implementations, the instrument guide 566 may include one or more guide features (e.g., cutting surfaces) 595 for resecting a bone B. The alignment guide 520 may be positioned relatively close to the cutting surfaces 595 to reduce a likelihood that captured images of the instrument guide 566 and/or anatomy A may be skewed.

The novel devices and methods of this disclosure provide improved accuracy in positioning imaging devices relative to an anatomy of the patient. The imaging device may capture one or more images having a field of view that may be substantially perpendicular to an alignment guide, which may improve accuracy in removing bone and other tissue from the anatomy and positioning implant components based on a surgical plan for the patient, which may improve mobility. The disclosed techniques may be useful in confirming a depth of alignment relative to the camera field of view, which may be established by alignment between the foreground and background sights of the alignment device. The alignment device may be positioned along, or otherwise adjacent to, the optical center of the camera, which may facilitate comparing the captured image to a virtual depiction associated with the surgical plan. The alignment of patient-specific and/or configurable (e.g., calibrated) surgical instruments (e.g., guides) may be confirmed according to the surgical plan based on the captured images. The disclosed techniques may be utilized to reduce the amount of imaging required and exposure to the patient and may reduce the time of the operation.

Although the different non-limiting embodiments are illustrated as having specific components or steps, the embodiments of this disclosure are not limited to those particular combinations. It is possible to use some of the components or features from any of the non-limiting embodiments in combination with features or components from any of the other non-limiting embodiments.

It should be understood that like reference numerals identify corresponding or similar elements throughout the several drawings. It should further be understood that although a particular component arrangement is disclosed and illustrated in these exemplary embodiments, other arrangements could also benefit from the teachings of this disclosure.

The foregoing description shall be interpreted as illustrative and not in any limiting sense. A worker of ordinary skill in the art would understand that certain modifications could come within the scope of this disclosure. For these reasons, the following claims should be studied to determine the true scope and content of this disclosure.

1. An alignment guide for surgical imaging comprising:
a guide body including first and second sidewalls interconnecting proximal and distal portions to bound a cavity;
wherein the proximal portion includes a foreground sight, the distal portion includes a background sight, and the foreground sight is visually alignable with the background sight to indicate an orientation of the alignment guide relative to a field of view of an imaging device; and
wherein a first alignment opening extends along a first reference plane between the first sidewall and the cavity, a second alignment opening extends along a second reference plane between the second sidewall and the cavity, and the first and second reference planes are transverse to each other and intersect along the background sight.

2. The alignment guide as recited in claim 1, wherein the guide body is securable to an instrument guide.

3. The alignment guide as recited in claim 1, wherein the first and second reference planes are substantially perpendicular to each other.

4. The alignment guide as recited in claim 1, wherein:
the first and second alignment openings extend from a proximal face of the proximal portion; and
an arc shaped channel extends about a periphery of the foreground sight to interconnect the first and second alignment openings along the proximal face.

5. The alignment guide as recited in claim 1, wherein the background sight includes first and second alignment members that intersect at a junction to establish a cross-shaped geometry.

6. The alignment guide as recited in claim 5, wherein the first and second reference planes follow the respective first and second alignment members and intersect the junction.

7. The alignment guide as recited in any preceding claim 1, wherein:
the foreground sight includes an elliptical body situated in an elliptical opening along a proximal face of the proximal portion; and
the elliptical body includes a cross-shaped opening alignable with a central portion of the background sight.

8. The alignment guide as recited in claim 7, wherein the first and second reference planes intersect along the cross-shaped opening and the central portion.

9. The alignment guide as recited in claim 1, further comprising:
one or more guide passages dimensioned to receive a respective guide element.

10. The alignment guide as recited in claim 9, wherein the one or more guide passages include first and second guide passages that extend along respective passage axes, and the passage axes are substantially parallel to each other.

11. (canceled)

12. The alignment guide as recited in claim 9, wherein the one or more guide passages include first and second guide passages, and further comprising:
a bridge member interconnecting the guide body and a locating member, the bridge member dimensioned to span across a joint of an anatomy;
wherein the first guide passage is established in the guide body, and the second guide passage is established in the locating member.

13. (canceled)

14. The alignment guide as recited in claim 1, wherein the guide body comprises a metallic material.

15. A surgical kit comprising:
an instrument guide including one or more apertures dimensioned to guide a surgical device, the instrument guide adapted to contact an anatomical surface of a patient based on a surgical plan; and
an alignment guide securable to the instrument guide at one or more interfaces, the alignment guide comprising:
a guide body including a cavity, a foreground sight and a background sight on opposite sides of the cavity, and first and second alignment openings interconnected by the cavity;
wherein the first and second alignment openings extend along respective reference planes that intersect at the foreground and background sights to establish respective pathways for a set of laser beams projectable onto the background sight; and
wherein the foreground and background sights are visually alignable with each other to indicate an orientation of the instrument guide relative to a field of view of an imaging device.

16. The surgical kit as recited in claim 15, wherein:
the one or more interfaces include a first interface and a second interface, the guide body is situated at a first orientation relative to the instrument guide at the first interface, and the guide body is situated at a second orientation relative to the instrument guide at the second interface.

17. The surgical kit as recited in claim 16, wherein:
the foreground and background sights are established along an axis of the guide body, and a position of the axis associated with the first orientation is substantially perpendicular to a position of the axis associated with the second orientation when projected onto a common reference plane.

18. (canceled)

19. A system for surgical imaging comprising:
an imaging device including a set of lasers operable to emit laser beams along respective paths oblique to a field of view of the imaging device such that the laser beams intersect each other adjacent to an optical center of the field of view; and
an alignment guide comprising:
a radiopaque guide body;
a foreground sight and a background sight visually alignable with each other to indicate an orientation of the guide body relative to the field of view of the imaging device; and
a set of alignment openings along an exterior of the guide body;
wherein the alignment openings extend along respective reference planes that intersect the foreground sight and the background sight, and the alignment openings are dimensioned to permit a projection of the respective laser beams onto the background sight.

20. The system as recited in claim 19, wherein:
the reference planes are substantially perpendicular to each other; and
the reference planes intersect adjacent to a center of the foreground sight and intersect adjacent to a center of the background sight.

21. The system as recited in claim 19 or 20, further comprising:
an instrument guide adapted to contact an anatomical surface of a patient based on a surgical plan;
wherein the alignment guide is securable to the instrument guide to indicate an orientation of the instrument guide relative to the field of view of the imaging device.

22. The system as recited in claim 19, wherein the guide body is radiopaque such that visibility of the background sight is partially obstructed adjacent to the alignment openings.

23. The system as recited in claim 19, wherein the imaging device includes a fluoroscopic camera.

24-39. (canceled)
